SEQUENCE LISTING (1 sequence)

SEQ ID NO: 1 - protein, 553 aa, Cypridina noctica
MKTLILAVALVYCATVHCQDCPYEPDPPNTVPTSCEAKEGECIDSSCGTCTRDILSDGLCENKPGKTCCRMCQYVIECRVEAAGWFRTFYGKRFQFQEPGTYVLGQGTKGGDWKVSITLENLDGTKGAVLTKTRLEVAGDIIDIAQATENPITVNGGADPIIANPYTIGEVTIAVVEMPGFNITVIEFFKLIVIDILGGRSVRIAPDTANKGMISGLCGDLKMMEDTDFTSDPEQLAIQPKINQEFDGCPLYGNPDDVAYCKGLLEPYKDSCRNPINFYYYTISCAFARCMGGDERASHVLLDYRETCAAPETRGTCVLSGHTFYDTFDKARYQFQGPCKEILMAADCFWNTWDVKVSHRNVDSYTEVEKVRIRKQSTVVELIVDGKQILVGGEAVSIPYSSQNTSIYWQDGDILTTAILPEALVVKFNFKQLLVVHIRDPFDGKTCGICGNYNQDFSDDSFDAEGACDLTPNPPGCTEEQKPEAERLCNSLFAGQSDLDQKCNVCHKPDRVERCMYEYCLRGQQGFCDHAWEFKKECYIKHGDTLEVPDECK

(19) United States
(12) Patent Application Publication
(10) Pub. No.: US 2010/0120075 A1
(43) Pub. Date: May 13, 2010

(54) Composition and a method for suppressing the luminescence intensity decline in a luciferin-luciferase reaction

(76) Inventors: Yoshihiro Ohmiya, Ikeda-shi (JP); Chun Wu, Ikeda-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/292,085

(22) Filed: Nov. 12, 2008

(51) Int. Cl. C12Q 1/66

(52) U.S. Cl. 435/8

(57) ABSTRACT

The invention relates to compositions and methods for suppressing the luminescence intensity decline that occurs as the reaction between Cypridina luciferase and luciferin proceeds.

A composition and a method for suppressing the luminescence intensity decline in a luciferin-luciferase reaction

### FIELD OF INVENTION

The present invention relates to compositions and methods for suppressing the luminescence intensity decline that occurs as the reaction between Cypridina luciferase and luciferin proceeds.

### BACKGROUND OF THE INVENTION

Luciferase is an enzyme that catalyzes the oxidation of luciferin, which results in light emission. As such, luciferases are known as light-producing enzymes. A variety of luciferases are known from many organisms including firefly beetles (Photinus pyralis), sea pansies (Renilla), copepods, oplophorus and so on. Different luciferases have different structures and propeties (such as, for example, substrate specificity, stability, color of the light, luminecence intensity etc.).

Light from the luciferase-luciferin reaction can be detected by light-sensitive apparatuses such as luminometers and special optical microscopes. This makes luciferase a good reporter, enabling real-time observation of biological processes in living cells and organisms. Luciferases are therefore extensively employed in the field of biological research. A major application of luciferase-lufirerin system are reporter assays, which can be used to evaluate the effects of foreign factors such as drug candidates, analyze intracellular signal transduction, and observe the expression of target genes. Reporter assays are also used to quantitatively measure target substances such as proteins in organisms.

For these and other applications, many luciferases, including recombinant ones, have been used. Cypridina luciferase is one of the most commonly used luciferases because it has good stability at the temperature and pH of the human body, and is secreted when produced in E. coli, insect cells or mammalian cells.

Although, luciferases are useful enzymes, one of their drawbacks is that the intensity of the luminescence declines as the luciferase-luciferin reaction proceeds. This is particularly problematic when a reporter assay using a luciferase is carried out on a 96-well plate. Since it usually takes more than ten minutes for even a person skilled in the art to distribute luciferin or luciferase to each well one by one, there is a gap between the wells with respect to the time at which the luciferin-luciferase reaction and the decline start. The light intensity decreases rather drastically in the beginning of the reaction, which prevents a direct comparison of the light intensity among the wells at a certain point in time of the reaction. Therefore, suppressing the decline in the luminescent intensity during the reaction time is desired.

With respect to this problem, WO 2006/096735 A2 disclosed that nonionic surfactants NP-40 and Triton X-100 suppressed a reaction time-dependent decline in the luminescent intensity from Gaussia luciferase, while an anionic surfactant, sodium dodecyl sulfate, did not suppress the decline, but deactivated the luciferase (see, for example, FIG. 2). These surfactants worked similarly for Renilla luciferase (see FIG. 6).

### Problems to be Solved by the Invention

An object of the invention is to provide the means to suppress a time-dependent decline in the luminescence intensity emitted from the reaction between Cypridina luciferase and luciferine.

### Means for Solving the Problems

Having conducted extensive research, the inventors found, surprisingly, that unlike with Gaussia luciferase, anionic surfactants do not deactivate Cypridina luciferase; and, more surprisingly, that the surfactants can even suppress a decline in the luminescence intensity that occurs as the Cypridina luciferase-luciferin reaction proceeds. Further studies have led inventors to discover that said suppressing effect can be significantly enhanced when a nonionic surfactant is present in the reaction solution, together with an anionic surfactant. Based on these discoveries and further research, the inventors have arrived at the following inventions.

1. A composition for suppressing a time-dependent decline in the intensity of luminescence emitted by Cypridina luciferase, comprising at least one anionic surfactant.

2. The composition of item 1, further comprising at least one nonionic surfactant.

3. The composition of item 1, wherein the at least one anionic surfactant is sodium dodecyl sulfate.

4. The composition of item 1, wherein the at least one nonionic surfactant is nonyl phenoxylpolyethoxylethanol (NP-40) and/or polyoxyethylene octyl phenyl ether (Triton X-100).

5. A method for suppressing a time-dependent decline in the intensity of luminescence emitted by Cypridina luciferase, wherein Cypridina luciferase is reacted with luciferin in the presence of at least one anionic surfactant.

6. The method of item 5, wherein at least one nonionic surfactant is further present in the reaction between Cypridina luciferase and luciferin.

7. The method of item 5, wherein the at least one anionic surfactant is sodium dodecyl sulfate.

8. The method of item 6, wherein the at least one nonionic surfactant is nonyl phenoxylpolyethoxylethanol (NP-40) and/or polyoxyethylene octyl phenyl ether (Triton X-100).

9. An agent for suppressing a time-dependent decline in the intensity of luminescence emitted by Cypridina luciferase, comprising at least one anionic surfactant.

### BRIEF DESCRIPTION OF THE DRAWINGS

FIG. 1 shows patterns of the decline in the luminescent intensity that occurred as the light-producing reaction between Cypridina luciferase and luciferin proceeds. The numbers in the figure correspond to the numbers of the buffers prepared in Example 1.

FIG. 2 shows a pattern of the decline in the luminescent intensity that occurred in an inventive buffer solution containing 10 ng/ml Cypridina luciferase, 0.2 wt % SDS, and 0.2 wt % NP 40 in 0.1 M Tris-HCl (pH 7.4) on a 96-well plate.

FIG. 3 shows a pattern of the decline in the luminescent intensity that occurred in a control buffer (0.1 M Tris-HCl (pH 7.4) with no surfactant) on a 96-well plate.

### DETAILED DESCRIPTION OF THE INVENTION

One embodiment of the invention is a composition for suppressing a time-dependent decline in the intensity of luminescence emitted by Cypridina luciferase, comprising at least one anionic surfactant. Another embodiment of the invention is a method for suppressing a time-dependent decline in the intensity of luminescence emitted by Cypridina luciferase, wherein Cypridina luciferase is reacted with luciferin in the presence of at least one anionic surfactant.

Cypridina luciferase is a monomeric, naturally secreted, 62-kDa glycoprotein that can be expressed in and secreted from transformed mammalian cells. Unlike firefly luciferase, which requires ATP (and for sustained activity, coenzyme A), Cypridina luciferase uses no cofactors other than water and O₂ SO as to oxidize luciferin to produce light. Its luminescent reaction proceeds optimally at pH 7.2 and physiological salt concentrations. These characteristics makes Cypridina luciferase an suitable luciferase for biological assays of mannmalian cells.

The amino acid sequece of the wild-type Cypridina luciferase is shown as SEQ ID NO: 1 in the attached sequence listing. The base sequence of Cypridina luciferase is available at the NCBI database under the Accession Nos. AAB86460, AAA30332, and BAD08210. A vector containing the Cypridina luciferase base sequence is also commecially available, for example, from ATTO Corperation (Tokyo). Based on this information, the luciferase can be readily obtainable according to genetic engineering techniques known in the art. Recombinant Cypridina luciferases can also be used as long as the enzymes are capable of catalyzing luciferin oxidation to produce light. Recombinant luciferases can be obtained according to genetic engineering techniques known in the art, such as site-specific mutagenesis. This recombinant Cypridina luciferase preferably has 85% or higher amino acid sequence homology to the wild-type sequence. More preferably, this recombinant Cypridina luciferase has 90% or higher amino acid homology to the wild-type sequence. Even more preferably, this recombinant Cypridina luciferase has 95% or higher amino acid homology to the wild-type sequence. Most preferably, this recombinant Cypridina luciferase has 98% or higher amino acid sequence homology to the wild-type sequence.

In regards to the invention, luciferin is a substrate of Cypridina luciferase, and produces light as a result of the oxidative reaction. A preferable example of the luciferin is Cypridina luciferin, which is produced by Cypridina noctica, and has the structure as shown below.

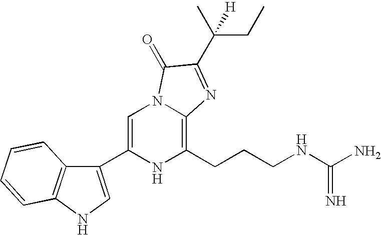

Cypridina luciferin is commercially available, for example, from ATTO Corporation (Tokyo). Luciferin analogs can also be used, so long as luminescence takes place following the oxidation that is catalyzed by Cypridina luciferase. Such luciferin analogs are described by Wu et al. (Tetrahedron Letters, 47, 753, 2006). Cypridina luciferin and its analogs can be synthesized according to Wu et al. Tetrahedron Letters, 47, 753, (2006).

Cypridina luciferase produces light when it catalyzes the oxidation of luciferin. More specifically, Cypridina luciferase catalyzes the oxidative reaction of luciferin to produce excited luciferin, and light is emitted when the excited luciferin is returned to a non-excited state. The intensity of luminescence emitted by Cypridina luciferase is the intensity of light emitted as a result of said reaction catalyzed by Cypridina luciferase.

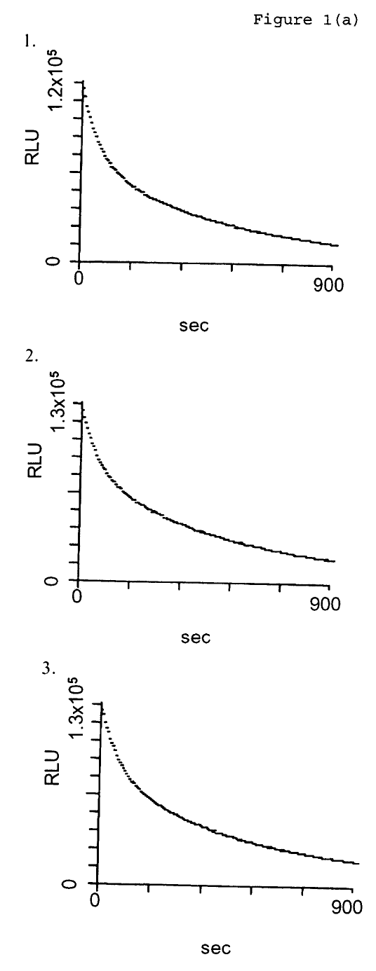

The time-dependent decline in the intensity of luminescence is a decline in the luminescence intensity that occurs as the reaction between Cypridina luciferase and luciferin proceeds. The decline usually starts immediately after Cypridina luciferase and luciferin are mixed in a solution, and the rate of the decline is usually not constant or proportional to the reaction time. Therefore, the time-dependent decline is not limited to a decline proportional to the reaction time, and can be any decline in the light intensity that occurs as the reaction progresses with time. In fact, a relatively sharp drop takes place in the beginning of the reaction, followed by a more gradual decrease as the reaction proceeds, as shown in FIG. 1. The cause of the decline is not yet well understood, but is probably due to the product inhibition of the oxidative reaction. As the oxidation of luciferin proceeds, oxyluciferin accumulates in a reaction solution, which is believed to slow down the reaction, resulting in a decline in the light intensity. The decline is observed when an overly large amount of luciferin is provided. Therefore, the decline is not due to a lack of the substrate.

The time-dependent decline in the luminescent intensity can be suppressed by the inventive composition comprising at least one anionic surfactant. Specifically, the decline can be suppressed by having at least one anionic surfactant present in the catalytic reaction of Cypridina luciferase and luciferin. Anionic surfactants that can be used in the invention are not particularly limited, so long as the surfactants have the ability to suppress the time-dependent decline. Examples of the anionic surfactants include carboxyl acid salts such as sodium cholate, sodium laurate, sodium myristate, sodium N-lauroylsacosinate, sodium oleate, sodium palmitate, and sodium stearate; sulfonic acid salts such as sodium 1-pentadecanesulfonic acid, sodium 1-dodecanesulfonate, sodium 1-hexadecanesulfonate, sodium 1-octadecanesulfonate, sodium 1-tridecanesulfonate, di(2-ethylhexyl)sulfosuccinate, sodium dimethyl 5-sulfoisophthalate, and sodium dodecylbenzenesulfonate; phosphoric acid salts such as sodium monododecyl phosphate; and sulfuric acid ester salts such as sodium dodecyl sulfate (SDS), and sodium hexadecylsulfate. These anionic surfactants are commercially available. The inventive composition may contain a single anionic surfactant, or several anionic surfactants used in combination. Preferable anionic surfactants are those categorized as salts of sulfuric acids ester, and the most preferable anionic surfactant is sodium dodecyl sulfate.

In the present invention, an anionic surfactant can be used by itself to suppress the time-dependent decline in the intensity, without being combined with other substances. In such an embodiment of the invention, the anionic surfactant may be recognized as an agent for suppressing a time-dependent decline in the intensity of luminescence emitted by Cypridina luciferase. Cypridina luciferase has, in addition to sugar chains, 47 Cysteins and 17 disulfide bonds in the polypeptide. These structural features probably attribute to the good stability of the enzyme. It is considered that due to such structures and the stability, Cypridina luciferase does not lose activity in the presence of a relatively strong surfactant like SDS, and is rather protected from being in contact with oxyluciferin.

In a preferred embodiment of the invention, the inventive composition comprising at least one anionic surfactant further comprises at least one nonionic surfactant. Suppression of the time-dependent decline can be enhanced by having at least one anionic surfactant and at least one nonionic surfactant present in the Cypridina luciferase-luciferin reaction. Nonionic surfactants that can be used in the invention are not particularly limited, as long as the surfactants can suppress the time-dependent decline in the luminescence intensity in combination with at least one anionic surfactant. Preferred anionic surfactants are those that improve the suppression effect of anionic surfactants. Examples of nonionic surfactants are ester-ether surfactants such as polyethylene sorbitan monolaurate (Tween 20), polyoxyethylene sorbitan monopalmitate (Tween 40), polyoxyethylene sorbitan monostearate (Tween 60), polyoxyethylene sorbitan monooleate (Tween 80), and polyoxyethylene sorbitan trioleate (Tween 85); ester surfactants such as monomyristin, monostearin, monopalmitin, polyethylene glycol monostearate, sorbitan monolaurate (Span 20) sorbitan monopalmitate (Span 40), sorbitan monostearate (Span 60), sorbitan monooleate (Span 80), sorbitan sesquioleate (Span 83), and sorbitan trioleate (Span 85); and ether surfactants such as diethylene glycol monododecyl ether, ethylene glycol monododecyl ether, polyethylene glycol mono-4-octylphenyl ether, tetraethylene glycol monododecyl ether, triethylene glycol monododecyl ether, polyoxyethylene octyl phenyl ether (Triton X-100), and polyethyleneglycol-p-isooctylphenyl ether (NP-40). These and other nonionic surfactants that can be used in the invention are commercially available. Preferred nonionic surfactants are ether surfactants, and more preferred anionic surfactants are Triton X-100 and NP40. The inventive composition may comprise a single nonionic surfactant, or several nonionic surfactants used in combination.

When the inventive composition comprising at least one anionic surfactant comprises at least one nonionic surfactant, any one or more anionic surfactants can be combined with any one or more nonionic surfactants at any ratio, as long as the composition suppresses the time-dependent decline in the luminescent intensity. Preferable combinations of anionic surfactants and nonionic surfactants are combinations of one or more anionic surfactants selected from sulfuric acid ester salts surfactants and one or more nonionic surfactants selected from ether surfactants. More preferable combinations of anionic surfactant and nonionic surfactant are combinations of SDS and any one or more nonionic surfactants selected from ether surfactants. Further more preferable combinations are the combination of SDS and Triton X-100, and the combination of SDS and NP-40. Preferable weight ratio of the total anionic surfactants to the total nonionic surfactants in the inventive composition is 1:1 to 1:10, more preferably 1:1 to 1:5.

In a preferred embodiment of the invention, the inventive composition comprising at least one anionic surfactant and optionally comprising at least one nonionic surfactant further comprises at least one calcium compound. By having calcium ions present in the Cypridina luciferase-luciferin reaction together with at least one anionic surfactant, and optionally at least one nonionic surfactant, the luminescent intensity is intensified probably because calcium ions activates the enzyme. Calcium compounds are calcium-comprising compounds that release calcium ions in a solution. Calcium compounds can intensify the luminescent intensity. Calcium compounds that can be used in the invention are available in. the market; examples thereof include nonorganic calcium compounds such as calcium oxide, calcium hydroxide, calcium chloride, calcium phosphate, monobasic calcium phosphate, dibasic calcium phosphate, tribasic calcium phosphate, calcium hypophosphate, and calcium sulfate; and organic calcium compounds such as calcium carbonate, calcium citrate, calcium malate, calcium lactate, calcium formate, calcium gluconate, calcium glycerophosphate, calcium levulinate, and calcium tartrate. Preferred calcium compounds are non-organic calcium compounds, and more preferred calcium compounds are calcium chloride. The inventive composition may comprise a single calcium compound or several calcium compounds used in combination, so long as the luminescence can be enhanced.

The inventive composition may comprise other components such as BSA that are normally added to a reaction solution for a luciferase-luciferin reaction.

The inventive composition comprising at least one anionic surfactant may be prepared in powder form or in solution form. The powder-form composition may be formulated according to procedures commonly used in the art. For example,. the inventive composition may be prepared by mixing at least one anionic surfactant, at least one nonionic surfactant and at least one calcium compound. When the inventive composition takes powder form, the composition can be added to a solution (e.g., a buffer) for a Cypridina luciferase-luciferin reaction, so as to suppress the decline in the luminescence intensity over time.

The inventive composition can be added to a reaction solution at any concentration so long as the time-dependent decline in the luminescence is suppressed. Preferably, the inventive composition is added to a reaction solution so that the solution contains anionic surfactant at 0.01 to 1.0 wt %, more preferably at 0.1 to 0.5 wt %, nonionic surfactant at 0.01 to 1.0 wt %, more preferably at 0.1 to 0.5 wt %, and calcium ion at 1 to 300 mM, more preferably at 10 to 50 mM. Such reaction solutions are some of the preferred embodiments of the inventive composition.

### EFFECT OF THE INVENTION

In accordance with the invention, the time-dependent decline in the luminescent intensity can be suppressed. In one embodiment of the invention, preferably 50% or more, more preferably 60% or more, further more preferably 70% or more, even more preferably 80% or more of the luminescent intensity is retained three minutes from the start of the reaction, compared to the initial intensity. In another embodiment of the invention, preferably 14% or more, more preferably 30% or more, further more preferably 50% or more, even further more preferably 60% or more of the luminescent intensity is retained 15 minutes from the start of the reaction, compared to the initial luminescent intensity.

The present invention is further explained with reference to the following Examples. However, it should be understood that the invention is not limited to the Examples.

### EXAMPLE 1

Eight different 0.1 M Tris-HCl buffers containing 10 ng/ml Cypridina luciferase and one or more surfactants were prepared in accordance with the following:

Buffers:

1. 0.1 M Tris-HCl pH7.4

2. 0.1 M Tris-HCl pH7.4/0.2% Triton X-100

3. 0.1 M Tris-HCl pH7.4/0.2% NP-40

4. 0.1 M Tris-HCl pH7.4/0.2% SDS

5. 0.1 M Tris-HCl pH7.4/0.2% SDS/0.2% Triton X-100

6. 0.1 M Tris-HCl pH7.4/0.2% SDS/0.2% NP-40

7. 0.1 M Tris-HCl pH7.4/0.2% SDS/0.2% Triton X-100/25 mM CaCl₂

8. 0.1 M Tris-HCl pH7.4/0.2% SDS/0.2% NP-40/25mM CaCl₂

Triton X-100, NP-40 and SDS were obtained from Wako Pure Chemical Industries, Ltd. The luciferase-luciferin reaction was initiated by adding luciferin to the respective buffer at a final concentration of 0.5 μM, and mixing. The intensity of the light produced from the reaction was measured against the reaction time using a luminometer. The results are shown in. Table 1 below, and in FIG. 1.

TABLE 1

| Buffer no. | half life (min) | I₃/I₀ | I₁₅/I₀ |
|---|---|---|---|
| 1 | 2.3 | 0.41 | 0.11 |
| 2 | 2.7 | 0.44 | 0.14 |
| 3 | 2.7 | 0.46 | 0.14 |
| 4 | 5.4 | 0.66 | 0.14 |
| 5 | 13.5 | 0.83 | 0.5 |
| 6 | 13.4 | 0.84 | 0.5 |
| 7 | 10.5 | 0.8 | 0.4 |
| 8 | 10.5 | 0.8 | 0.4 |

I₀: Light intensity measured immediately after luciferin was mixed with Cypridina luciferase
I₃: Light intensity measured 3 minutes after the start of the reaction
I₁₅: Light intensity measured 15 minutes after the start of the reaction

A comparison of the results for buffers 1 and 4 shown in Table 1 and FIG. 1 indicates that the luminescent intensity, which declined as the reaction proceeded, was suppressed or alleviated when the anionic surfactant SDS was present in the reaction. The suppression effect was enhanced when the nonionic surfactants Triton X-100 or NP-40 coexisted with SDS in the reaction solution (see buffers 5 and 6).

### EXAMPLE 2

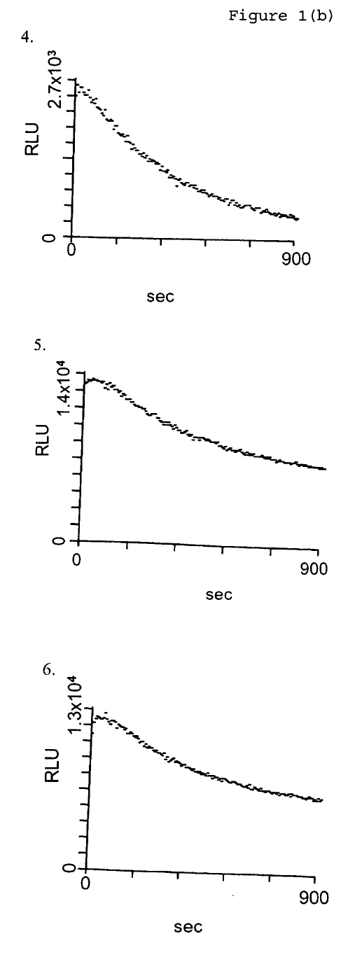

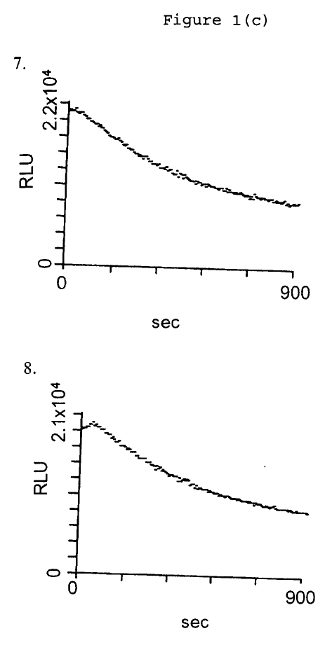

A buffer containing Cypridina luciferase at lOng/ml, 0.2 wt % SDS, and 0.2 wt % NP 40 in 0.1 M Tris-HCl (pH 7.4) was prepared as described in Example 1. A control buffer was prepared to contain 10 ng/ml Cypridina luciferase in 0.1 M Tris-HCl (pH 7.4) with no surfactant. Each buffer was separately distributed to each well of a 96-well plate, to which Cypridina luciferin was added at the final concentration of 0.5 μM in order from well (1A) to well (1H), then from well (2H) to well (2A), then from well (3A) to well (3H), until well (12A). The luminescent activity of each well was measured by luminometer after the luciferin was added to all of the wells. The integration period for each well was 20 seconds. FIG. 2 shows the results for the inventive composition (buffer) containing SDS and NP 40, and FIG. 3 shows the results for the control buffer containing no surfactant. A comparison of FIGS. 2 and 3 clearly reveals that the decline of luminescent intensity was suppressed for the inventive buffer, as compared to the control.

1. A composition for suppressing a time-dependent decline in the intensity of luminescence emitted by Cypridina luciferase, comprising at least one anionic surfactant.

2. The composition of claim 1, further comprising at least one nonionic surfactant.

3. The composition of claim 1, wherein the at least one anionic surfactant is sodium dodecyl sulfate.

4. The composition of claim 2, wherein the at least one nonionic surfactant is polyethyleneglycol-p-isooctylphenyl ether (NP-40) and/or polyoxyethylene octyl phenyl ether (Triton X-100).

5. A method for suppressing a time-dependent decline in the intensity of luminescence emitted by Cypridina luciferase, wherein Cypridina luciferase is reacted with luciferin in the presence of at least one anionic surfactant.

6. The method of claim 5, wherein at least one nonionic surfactant is further present in the reaction between Cypridina luciferase and luciferin.

7. The method of claim 5, wherein the at least one anionic surfactant is sodium dodecyl sulfate.

8. The method of claim 6, wherein the at least one nonionic surfactant is polyethyleneglycol-p-isooctylphenyl ether (NP-40) and/or polyoxyethylene octyl phenyl ether (Triton X-100)

9. An agent for suppressing a time-dependent decline in the intensity of luminescence emitted by Cypridina luciferase, comprising at least one anionic surfactant.
